(19) United States
(12) Plant Patent Application Publication
(10) Pub. No.: US 2007/0028337 P1
(43) Pub. Date: Feb. 1, 2007

(54) Chrysanthemum plant named 'Ivory Time Lemon'

(76) Inventor: Ruth Parham, Bognor Regis (GB)

Correspondence Address: WINSTON & STRAWN LLP;PATENT DEPARTMENT, 1700 K STREET, N.W., WASHINGTON, DC, 20006, US

(21) Appl. No.: 11/488,589

(22) Filed: Jul. 18, 2006

Related U.S. Application Data
(60) Provisional application No. 60/703,338, filed on Jul. 28, 2005.

(51) Int. Cl. A01H 5/00

(52) U.S. Cl. PLT/289

(57) ABSTRACT

A new variety of Chrysanthemum plant named ‘Ivory Time Lemon,’ having a domed capitulum of lemon decorative flowers, dark glossy foliage and an even branching habit.

This application claims the benefit of U.S. Provisional Application No. 60/703,338, filed Jul. 28, 2005.

### LATIN NAME OF THE GENUS AND SPECIES

Botanical classification: Chrysanthemum morifolium.

### VARIETY DENOMINATION

The new Chrysanthemum variety denomination is ‘Ivory Time Lemon’.

### BACKGROUND OF THE INVENTION

The present invention comprises a new and distinct cultivar of Chrysanthemum botanically known as Chrysanthemum morifolium, and referred to by the cultivar name ‘Ivory Time Lemon’.

‘Ivory Time Lemon’, identified as 20193-1 was discovered as a whole plant natural mutation in a controlled cultivated planting of the variety ‘Ivory Time’ (unpatented seedling).

The new variety ‘Ivory Time Lemon’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Ivory Time Lemon’ is a pot type of Chrysanthemum plant variety having a domed capitulum form of lemon decorative flowers.

### Comparison with Original Cultivar

Plants of the new Chrysanthemum variety ‘Ivory Time Lemon’ are similar to the original cultivar ‘Ivory Time’ in plant habit and growth rate. In side-by-side comparisons in Chichester, West Sussex, United Kingdom, under commercial practice, plants of the new Chrysanthemum variety ‘Ivory Time Lemon’ differed from plants of the original cultivar ‘Ivory Time’ in the following characteristics:
1. The new variety ‘Ivory Time Lemon’ produces lemon decorative flowers whereas the original cultivar ‘Ivory Time’ produces white decorative flowers.
2. Plants of the new variety ‘Ivory Time Lemon’ have a quicker response time than the plants of the cultivar ‘Ivory Time’.

### Comparison with ‘Party Time’

Plants of the new Chrysanthemum variety ‘Ivory Time Lemon’ are dissimilar to the cultivar ‘Party Time’ (U.S. Plant Pat. No. 10,896) in plant habit and growth rate. However, in side-by-side comparisons in Chichester, West Sussex, United Kingdom, under commercial practice, plants of the new Chrysanthemum variety ‘Ivory Time Lemon’ differed from plants of the cultivar ‘Party Time’ in the following characteristics:
1. The new variety ‘Ivory Time Lemon’ produces lemon decorative flowers whereas the cultivar ‘Party Time’ produces white decorative flowers.
2. Plants of the new variety ‘Ivory Time Lemon’ have smaller and shorter inflorescence than plants of the cultivar ‘Party Time’.
3. Plants of the new variety ‘Ivory Time Lemon’ have a quicker response time when compared to the plants of the cultivar ‘Party Time’.

### BRIEF DESCRIPTION OF ILLUSTRATIONS

Typical specimens of the plant and flowers for the new Chrysanthemum variety ‘Ivory Time Lemon’ are shown in the accompanying digital photograph. The colors shown are as true as possible within the usual limits of this kind of illustration.

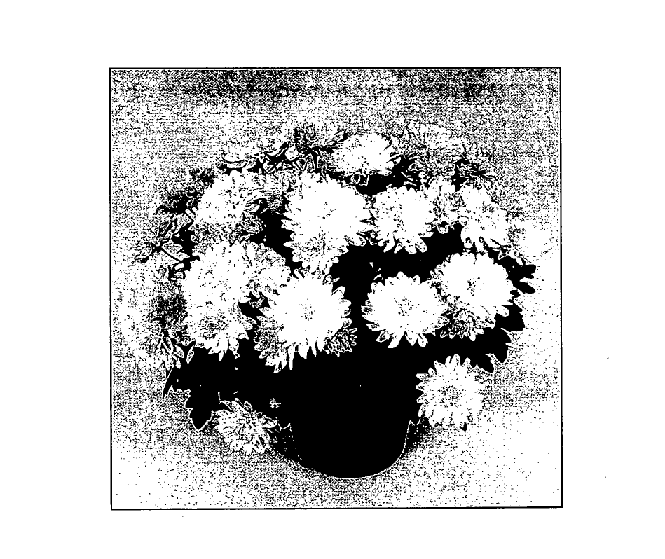

FIG. 1 is a whole plant view of the new variety ‘Ivory Time Lemon’ grown in a pot. The plant shown in the illustration is 55 days from the commencement of Short Days.

### DETAILED BOTANICAL DESCRIPTION

The following description of the new Chrysanthemum variety ‘Ivory Time Lemon’ is of plants grown in a greenhouse in Chichester, West Sussex, United Kingdom in the month of October. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 19° C. at night and about 17° C. to 24° C. during the day under light conditions of about 2,500 Lux. The plants respond well to the use of growth retardant, such as one B9 treatment at about 2 gram per liter. To produce a commercial product the plants may be pinched once with the center bud removed. The typical container size for commercial growth is 1 liter. It has been observed that the shelf life of the new variety is about 21 days with a response time of about 7 weeks. The new variety is suitable for growth in a temperature range of 15° C. to 25° C.

The following description is with respect to a plant produced as pot. In the description of this new Chrysanthemum variety, color values have been taken from The Royal Horticultural Society Colour Chart (R.H.S.C.C.).

### Plant

Plant type: pot.
Habit: upright.
Height (cm): 24.
Width (cm): 32.
Stem:
   Length (cm).—4.
   Diameter (cm).—0.5.
   Texture.—slightly pubescent.
   Color.—138B.
Branching characteristics: Upright and free branching.
Lateral Branches:
   Length (cm).—12.
   Diameter (cm).—0.5.
   Texture.—slightly pubescent.
Number of breaks from pinch: 5.
Response time: 46 days.
Vigor: medium.
Shelf life: 21 days.
Disease (susceptibility/resistance observed): None observed to date.
Pest (susceptibility/resistance observed): None observed to date.
Drought and temperature (susceptibility/resistance observed): None observed to date.
Growth retardant type and treatment: 3 applications of 2.5 gm/liter B9@2, 21 and 28 days after sticking unrooted cuttings.

The plants were grown for 2 weeks in Long Day conditions (20 hours of light) and then transferred to Short Day conditions (13 hours of dark).
Propagation:
   Type.—Vegetative Propagation Via Stem Cuttings.
   Time to rooting.—14 days with soil temperatures of 18° C.
   Rooting habit.—After 7 days the first roots emerge and form root primordia. In 14 long days a complete root system is developed.

### Foliage

Number of leaves per lateral branch: 16.
Compound or single: single.
Arrangement of leaves: alternate.
   Shape of leaf.—typically 5 lobed.
   Size of leaf.—Width (cm): 7 Length (cm): 8.5.
   Leaf apex.—acute.
   Base.—cordate.
   Attachment.—petioled.
   Aspect.—slightly undulating.
   Margin.—Palmately lobed.
   Surface characteristics.—Top: slightly pubescent. Bottom: pubescent.
Petiole:
   Color.—137C.
   Length (cm).—2.5.
   Diameter (cm).—0.3.
Venation:
   Color.—upper side: 138B. underside: 138B.
Color:
   Mature leaf, upper side.—137B; under side: 139C.
   Young leaf, upper side.—137A; under side: 137C.

### Flower

Flower appearance: matte.
Flower type: decorative.
Flower form: domed.
Flower shape: round.
Flowering habit: domed capitulum.
Number of blossoms per branch: 8.
Typical and observed flowering season: January to December.
Inflorescence form: cyme.
   Depth of fully expanded blossoms (cm).—1.5.
   Diameter of fully expanded blossoms (cm).—6.
Phyllaries:
   Number.—14.
   Color.—137C.
   Length (cm).—1.
   Width (cm).—0.2.
   Texture/appearance.—slightly pubescent.
Peduncle:
   Length (cm).—12.
   Diameter (cm).—0.5.
   Angle from stem (degrees).—10.
   Color.—138B.
   Surface.—slightly pubescent.
   Habit.—slightly undulating.
   Strength.—medium.
Pedicel:
   Length.—terminal (cm): 4.5; lateral (cm): 5.5.
   Diameter (cm).—0.2.
   Color.—138B.
   Surface.—slightly pubescent.
   Habit.—slightly undulating.
   Strength.—medium.
Ray florets:
   Form/shape.—straight.
   Texture/appearance.—matte.
   Number per flower.—Approximately 100.
   Length (cm).—2 Width (cm): 0.5.
   Apex.—rounded.
   Base.—tapered.
   Margin.—entire.
Disc florets:
   Form/shape.—cylindric.
   Texture/appearance.—shiny.
   Number per flower.—5.
   Length (cm).—0.3; Width (cm): 0.1.
   Diameter of disc (cm).—0.3.
Fragrance: none.
Flower bud (at onset of color):
   Length (cm).—1.
   Diameter (cm).—1.
   Form/shape.—globular.
General flower color:
   1. Ray florets, upper side.—Immature: 2C. Mature: 2D Older/Fading: 2D.
   2. Ray florets, under side.—Immature: 4D Mature: 4D Older/Fading: 4D.
   3. Disc florets.—Immature: 3B Mature: 3C Older/Fading: 3C.
   4. Bud.—2C.
Flower progression with age: flower form does not change with age and there is very little color fading.

### Reproductive Organs

Gynoecium present on ray and disc florets:
Ray florets: Pistillate.
   Pistil number.—100.
   Stigma color.—6C.
   Stigma shape.—forked.
   Style color.—6C.
   Style length (cm).—0.5 cm.
Disc florets: Pistillate.
   Pistil number.—5.
   Stigma color.—6C.
   Stigma shape.—forked.
   Style color.—6C.
   Style length (cm).—0.2.
Androecium: none observed.
Fruit and seeds: none observed.

What is claimed is:

1. A new and distinct variety of Chrysanthemum plant, substantially as described and illustrated herein.
